(19) United States
(12) Patent Application Publication
(10) Pub. No.: US 2012/0125792 A1
(43) Pub. Date: May 24, 2012

(54) DEVICES, KITS AND METHODS FOR SURGICAL FASTENING

(75) Inventor: Stephen D. Cassivi, Rochester, MN (US)

(73) Assignee: Mayo Foundation for Medical Education and Research, Rochester (US)

(21) Appl. No.: 13/062,908

(22) PCT Filed: Sep. 8, 2009
(86) PCT No.: PCT/US2009/056167

§ 371 Date: May 20, 2011

Related U.S. Application Data
(60) Provisional application No. 61/095,122, filed on Sep. 8, 2008.

(51) Int. Cl. A61B 17/068; B65D 81/24

(52) U.S. Cl. 206/205; 227/175.1

(57) ABSTRACT

Devices, kits and methods used to deliver provide radioactive material and a buttress element in a variety of procedures that typically involve the use of a stapler to fasten the buttress material to tissue are described herein. The radioactive material is provided to deliver radiation energy to the tissue. One or more medically useful agents may be provided in addition to the radioactive material.

### RELATED APPLICATION

The present application claims the benefit under 35 U.S.C. §119(e) of U.S. Provisional Patent Application No. 61/095,122, filed on Sep. 8, 2008 and titled METHODS AND DEVICES FOR SURGICAL FASTENING, which is hereby incorporated by reference in its entirety.

Devices, kits and methods for surgical fastening of anatomical tissue while delivering radiation energy are described herein.

Surgical fastening of anatomical tissue may be performed using, for example, a surgical fastening instrument (e.g., a surgical stapler). In some circumstances, it may also be desired to provide, for example, radiation to a localized region near surgically fastened anatomical tissue. Radiation may be provided in order to, for example, treat cancer.

In some medical procedures, localized application of radiation to anatomical tissue is desired. One method of applying radiation to anatomical tissue includes placing radioactive material (e.g., brachytherapy seeds) near the anatomical tissue to be irradiated. Brachytherapy is a form of radiotherapy wherein a radioactive source is sealed and placed in or near an area to be treated. When brachytherapy is planned, the brachytherapy seeds may be sewn by hand onto a piece of mesh. During a lung resection surgery, lung tissue is resected followed by the mesh (with the brachytherapy seeds thereon) being manually sutured to the non-resected lung tissue along the staple line. Such manual attachment of the brachytherapy seeds is time consuming and technically challenging.

### SUMMARY

Devices, kits and methods to simultaneously provide radioactive material (e.g., brachytherapy seeds) and a buttress element are described herein. The devices, kits and methods may be used in, for example, a surgical procedure (e.g., a lung resection). Methods of simultaneously providing radioactive material (e.g., brachytherapy seeds) and a buttress element are also provided. Codelivery of radioactive material and a buttress element may provide various advantages, such as reducing surgical time, reducing patient recovery time, and reducing technical difficulty of surgery. In some embodiments, a medically useful agent may be provided in addition to the radioactive material.

A buttress element may be a component of a surgical fastener system, which may include surgical fasteners including, but not limited to, staples. In one or more embodiments in which one or more staples are used as one or more surgical fasteners, a buttress element may be a staple line buttress element. Buttress elements may be used in, for example, surgical procedures that involve, for example, resection of anatomical tissue. Buttress elements may provide reinforcement of surgically fastened anatomical tissue at, for example, a staple line. See, e.g., Downey et al., “Functional Assessment of a New Staple Line Reinforcement in Lung Resection,” J. Surg. Res., 131 (1): 49-52 (March 2006). In one or more embodiments, buttress elements may provide increased hemostasis and aerostasis (reduction of air leaks) in surgically fastened anatomical tissue.

As used herein, anatomical tissue refers to any tissue of an animal (e.g., fish, amphibian, reptile, bird, or mammal) that may be surgically fastened (e.g., stapled). In one or more embodiments, the anatomical tissue includes lung tissue (e.g., diseased lung tissue) of which a portion may be or may have been resected. In one or more embodiments, the anatomical tissue may include tissues proximal to cancerous cells and/or tumors.

Buttress elements may be used in a variety of different procedures including, for example, lung resection surgery wherein portions of the lungs are surgically fastened with, for example, a surgical stapler. In the present disclosure, a wide variety of staplers may be used including, but not limited to, those adapted for use in endoscopic surgery, such as those disclosed in U.S. Pat. Nos. 5,597,107 (Knodel et al.) and 6,656,193 (Grant et al.) and those staplers designed for open procedures, such as those disclosed in U.S. Pat. No. 5,415,334 (Williamson, IV et al.) and PCT Publication No. WO 2008/057281 (Stopek).

In some embodiments, a kit for buttressing tissue at an internal body location as described herein includes one or more sheets of buttress material in a package, wherein the buttress material is configured for delivery and attachment to tissue using a stapler; and radioactive material in the package, wherein the radioactive material is configured for attachment to the one or more sheets of buttress material.

In some embodiments of the kits described herein, the radioactive material is provided integrally with the buttress material.

In some embodiments of the kits described herein, the radioactive material is incorporated into one or more fibers in the buttress material.

In some embodiments of the kits described herein, the radioactive material is separate from the buttress material.

In some embodiments of the kits described herein, the kit includes carrier material, and the radioactive material is provided integrally with the carrier material. In some embodiments, the radioactive material is incorporated into one or more fibers in the carrier material.

In some embodiments of the kits described herein, wherein the kit further includes staples. In some embodiments, the radioactive material is provided integrally with the staples.

In some embodiments of the kits described herein, the kit includes a medically useful agent. In some embodiments, the medically useful agent is provided integrally with the buttress material.

In some embodiments of the kits described herein, the kit further includes staples and carrier material, and the radioactive material is provided integrally with the carrier material. In some embodiments, the kit may also include a medically useful agent. In some embodiments, the medically useful agent is provided integrally with the buttress material. In some embodiments, the medically useful agent is provided integrally with the staples. In some embodiments, the medically useful agent is provided integrally with the carrier material.

In some embodiments, the surgical fastening devices described herein may include a buttress element and radioactive material attached to the buttress element.

In some embodiments of the surgical fastening devices described herein, the buttress element is attached to a surgical fastening instrument. In some embodiments, the surgical fastening device is a surgical stapler and the buttress element is adapted for delivery to a staple line formed by the surgical stapler.

In some embodiments of the surgical fastening devices described herein, the buttress element includes two or more layers of buttress material.

In some embodiments of the surgical fastening devices described herein, the radioactive material includes a radioactive isotope.

In some embodiments of the surgical fastening devices described herein, the radioactive material is a radioactive isotope of an element selected from the group consisting of iodine, palladium, or a combination thereof.

In some embodiments of the surgical fastening devices described herein, the radioactive material is a radioactive isotope selected from the group consisting of iodine-125, palladium-103, and combinations thereof.

In some embodiments of the surgical fastening devices described herein, the radioactive material includes a seed or a radioactive fiber.

In some embodiments of the surgical fastening devices described herein, the radioactive material is attached to a carrier element. In some embodiments, the carrier element is selected from the group consisting of vicryl mesh, polygluconate, bovine pericardium, small intestinal submucousa, and combinations thereof. In some embodiments, the carrier element is attached to the buttress element. In some embodiments, the radioactive material includes seeds attached to the carrier element. In some embodiments, the radioactive material is distributed on the carrier element to deliver a prescribed radiation dose.

In some embodiments of the surgical fastening devices described herein, the radioactive material is attached to the buttress element in a longitudinal configuration having a first axis having a dimension significantly longer than the dimensions in axes transverse to the first axis.

In some embodiments of the surgical fastening devices described herein, the carrier element is bound, and the bound carrier element may be rolled or folded. In some embodiments, the bound carrier element is mechanically bound with a binding device, wherein the binding device is optionally selected from the group consisting of a clamp, tie, hook, clip, or a combination thereof; and wherein the binding device is optionally removable from the carrier element; and further wherein the binding device may be manipulated to release the bound carrier element such that the carrier element may comprise a flat, sheet-like configuration.

In some embodiments of the surgical fastening devices described herein, the radioactive material is provided in two separate and discrete quantities.

In some embodiments, a method of buttressing a staple line and providing radiation therapy as described herein includes: attaching buttress material to selected tissue using staples; attaching radioactive material to the buttress material, wherein radiation therapy is provided to tissue proximate the buttress material.

In some embodiments of the methods described herein, a medically useful agent is provided proximate the buttress material.

The words “preferred” and “preferably” refer to embodiments that may afford certain benefits, under certain circumstances. However, other embodiments may also be preferred, under the same or other circumstances. Furthermore, the recitation of one or more preferred embodiments does not imply that other embodiments are not useful, and is not intended to exclude other embodiments from the scope of the invention.

As used herein, “a,” “an,” “the,” “at least one,” and “one or more” are used interchangeably. Thus, for example, a buttress element may be used to refer to one, two, three or more buttress elements.

The term “and/or” means one or all of the listed elements or a combination of any two or more of the listed elements.

The above summary is not intended to describe each embodiment or every implementation of the devices, kits and methods described herein. Rather, a more complete understanding of the devices, kits and methods described herein will become apparent and appreciated by reference to the following Description of Illustrative Embodiments and claims in view of the accompanying figures.

### BRIEF DESCRIPTIONS OF THE FIGURES

FIG. 1 depicts one embodiment of a device in the form of a stapler that may be used to deliver radioactive material as described herein.

FIG. 2 is an enlarged view of the jaws of the stapler of FIG. 1.

FIG. 3 is a block diagram depicting various components of a kit as described herein.

### DESCRIPTION OF ILLUSTRATIVE EMBODIMENTS

In the following description of illustrative embodiments, reference is made to the accompanying figures which form a part hereof, and in which are shown, by way of illustration, specific embodiments in which the devices, kits and/or methods may be practiced. It is to be understood that other embodiments may be utilized and structural changes may be made without departing from the scope of the present invention.

The devices. kits and methods described herein may be used to deliver provide radioactive material and a buttress element in a variety of procedures that typically involve the use of a stapler to fasten the buttress material to tissue, with the radioactive material being provided to deliver radiation energy to the tissue. In some embodiments, one or more medically useful agents may be provided in addition to the radioactive material.

In one or more embodiments, a stapler as used herein may include a handle for the operator to hold, an elongated shaft extending distally from the handle, two clamping members at the distal end of the elongated shaft, the two clamping members being movable from an open position to a closed position by operating a closure trigger attached to the handle. At least one of the two clamping members may include a staple cartridge that includes a plurality of staples. Also attached to the handle may be an actuator which, when actuated, expels a plurality of staples against the other clamping member in order to engage any material between the clamping members (e.g., anatomical tissue, buttress material, etc.).

In one or more embodiments, a buttress element may be used in conjunction with stapling anatomical tissue to, for example, potentially reduce bleeding of the stapled anatomical tissue and, when applied to lung tissue, to potentially reduce air leakage along the stapled lung tissue. In the present disclosure, a wide variety of buttress elements may be used, including, but not limited to, those mentioned in U.S. Pat. Nos. 5,468,253 (Bezwada et al.), 6,592,597 (Grant et al.), 6,656,193 (Grant et al.) and 6,704,210 (Myers), U.S. Patent Application Publication No. 2007/0246505 (Pace-Floridia et al.); and Downey et al., “Functional Assessment of a New Staple Line Reinforcement in Lung Resection,” J. Surg. Res., 131 (1): 49-52 (March 2006).

In the present disclosure, a buttress element may include one or more strips of buttress material having a staple line running longitudinally along the one or more strips. A buttress element may include two layers of buttress material, wherein each layer of buttress material may be disposed on one of the clamping members of a surgical stapler. The clamping members of the surgical stapler may then be manipulated into a closed position with anatomical tissue between the two layers of buttress material. The anatomical tissue may be surgically fastened by applying staples through the two layers of buttress material with the anatomical tissue therebetween. The plurality of staples may be applied such that the staples are arranged in one or more rows of staples. After the buttress element and anatomical tissue has been stapled with a plurality of staples, the buttress and anatomical tissue may be cut along the staple line along at least one row of staples (e.g., between two rows of staples) to sever one portion of anatomical tissue from another. Anatomical tissue may thereby be resected along a staple line of a buttress element.

In one or more embodiments of the present disclosure, a portion of the buttress element may be removed with resected tissue and another portion of the buttress element remains fastened to the non-resected tissue by, e.g., the line of staples. In the present disclosure, radioactive material may be attached to the buttress element fastening the anatomical tissue that is not resected. In other words, a buttress material having radioactive material attached thereto may be positioned between the clamping members of a surgical fastening instrument (e.g., surgical stapler) such that the radioactive material is present on at least one side of the clamping members (i.e., the side facing the non-resected tissue and opposite the side facing the tissue to be resected).

In the present disclosure, one or more layers of buttress material may be connected to form a single piece of buttress element. A buttress element may be formed by two or more layers of buttress material between the clamping members of a surgical stapler, such as that described in U.S. Patent Application Publication No. 2007/0246505. In the present disclosure, a “buttress element” refers to the one or more layers of buttress material fastened in a single actuation of staples therethrough and includes embodiments wherein a layer of buttress material is attached to another layer of buttress material and embodiments wherein a layer of buttress material is not attached to another layer of buttress material.

Any suitable buttress material may be used. Some potentially suitable buttress materials may include, e.g., natural or synthetic materials and may include, but are not limited to, bovine pericardium, collagen absorbable hemostat, vicryl (VICRYL® produced by Ethicon, Inc., Somerville, N.J.), ePTFE (expanded polytetrafluoroethylene), and those mentioned in U.S. Pat. Nos. 5,468,253 (Bezwada et al.), 6,592,597 (Grant et al.), 6,656,193 (Grant et al.) and 6,704,210 (Myers), U.S. Patent Application Publication No. 2007/0246505 (Pace-Floridia et al.); and Downey et al., “Functional Assessment of a New Staple Line Reinforcement in Lung Resection,” J. Surg. Res., 131 (1): 49-52 (March 2006). Those skilled in the art would recognize other suitable materials that may be used as a buttress material.

In one or more embodiments according to the present invention, one or more buttress elements may be used when, for example, surgically fastening a large amount of anatomical tissue. Further, more than one buttress material may be used in each of the one or more buttress elements.

In the present disclosure, a buttress element may include a carrier element. In one or more embodiments, a carrier element may be a portion of a buttress element or may be attached to a buttress element.

In the present invention, a carrier element may be constructed from a wide variety of carrier element materials. Any suitable carrier element material may be used. Some potentially suitable carrier element materials may include biocompatible carrier element materials to which radioactive material may be attached according to the present disclosure. Carrier element materials useful in the present invention may include, but are not limited to, meshes (polymeric or otherwise), fabrics (non-wovens, knits, wovens, etc.), films, etc.

In the present disclosure, a carrier element may be attached to a buttress element using any suitable technique. For example, a carrier element may be sewn to a buttress element, attached by adhesives, stapled, welded (chemically, thermally, etc.), or otherwise attached. One having ordinary skill in the art would recognize other manners in which a carrier element may be attached to a buttress element.

In the present disclosure, a buttress element having radioactive material attached thereto includes embodiments wherein the radioactive material is attached directly to the buttress element and embodiments wherein the radioactive material is attached to a carrier element that is attached to the buttress element.

In one or more embodiments, radioactive material (e.g., brachytherapy seeds) is attached to the carrier element. Any suitable technique for attaching the radioactive material to the carrier element may be used. Some potentially suitable attachment techniques may include, e.g., adhering, sewing, embedding, etc. Radioactive material may, for example, be provided in the form of a fibrous material that could, for example, constitute a portion of a carrier element attached to a buttress element or a portion of the buttress element.

In the present disclosure, radioactive material may be any suitable radioactive material for medical applications and include a wide variety of radioactive elements. In the present disclosure, medical applications for which radioactive material may be used include, but are not limited to, treatment of anatomical tissues, including, but not limited to cancerous cells and/or tumors, wherein the cancers may include lung cancer, thymic cancer, thymoma, metastatic cancers, metastatic sarcomas, etc.

Radioactive material may include, but is not limited to, brachytherapy seeds appropriate for a medical application. Brachytherapy seeds may be commercially available from Oncura (Arlington Heights, Ill.), BestMedical International Inc. (Springfield, Va.), and other suppliers. Suitable radioactive materials may include, but are not limited to, iodine (e.g., iodine-125), palladium (e.g., palladium-103), and combinations thereof. Some examples of brachytherapy seeds and some methods of using same may be found in, for example, Santos et al., “Comparison between sublobar resection and ¹²⁵Iodine brachytherapy after sublobar resection in high-risk patients with Stage I non-small-cell lung cancer,” Surgery 134 (4): 691-697 (October 2003); and Voynov et al., “Intraoperative ¹²⁵I Vicryl mesh brachytherapy after sublobar resection for high-risk stage I nonsmall cell lung cancer,” Brachytherapy 4 (4): 278-285 (December 2005). In the present disclosure, any suitable radioactive material may be chosen by one of skill in the art to, for example, provide an appropriate duration and/or intensity of radiation treatment.

The radioactive material is attached to the carrier element such that the amount of radioactive material and the distribution of radioactive material provide, for example, a prescribed dosage of radiation to anatomical tissue. For example, the radioactive material may be distributed on a carrier element in a regular or irregular pattern. In one or more embodiments, radioactive material (e.g., brachytherapy seeds) is arranged in a pattern wherein the radioactive material is regularly spaced in a repeating geometric pattern (e.g., parallelograms, triangles, circles, etc.). In one or more embodiments, the radioactive material is not evenly distributed across the carrier element. In embodiments including codelivery (e.g., to a staple line) of two or more carrier elements containing radioactive material, the distribution of radioactive material on one carrier element may be the same as the distribution on another carrier element, may be a mirror image of the distribution on another carrier element, or may be different than the distribution of radioactive material on another carrier element. Radioactive material attached to the buttress line may be distributed to provide radioactive effect to an appropriate margin of tissue around the resection margin/staple line.

In one or more embodiments in which the radioactive material includes brachytherapy seeds, it may be beneficial if the brachytherapy seeds are not located in the portion of the buttress element to be stapled. In other words, when surgically fastening the device of the present disclosure, it may be advantageous to avoid contacting brachytherapy seeds with staples.

In one or more embodiments, the radioactive material (e.g., brachytherapy seeds) is attached to the carrier element prior to attachment of the carrier element to the buttress element. In some embodiments, the radioactive material may be attached to the buttress element, either directly or attached via another material (e.g., a carrier element, etc.) before the buttress element is used as a surgical fastener (e.g., before the buttress element is stapled). The buttress element having radioactive material attached thereto may be positioned within the clamping members of a surgical fastening instrument (e.g., a surgical stapler) before the surgical fastening instrument is used to attach the buttress element along a resection line.

In one or more embodiments, the carrier element is adapted to be codelivered with a buttress element to a resection site (e.g., at a staple line). In one or more embodiments, the carrier element is adapted to be codelivered with a buttress element to a resection site in an endoscopic procedure. In such an embodiment, the carrier element is adapted to be codelivered with a buttress element through an endoscopic passage to a resection site.

In one or more embodiments, the carrier element may include a substantially flat, sheet-like material having two dimensions significantly longer than the third dimension (typically referred to as the thickness). Adapting the carrier element to be codelivered with a buttress element to a resection site may include rolling the carrier element into a roll having a longitudinal axis that is generally aligned along an edge of the clamping members of a stapler. Adapting the carrier element to be codelivered with a buttress element may also include folding the carrier element one or more times to reduce the length of at least one dimension generally transverse to the direction of the folds. In one or more embodiments, adapting the carrier element for codelivery with a buttress element may include binding the carrier element such that the bound carrier element has one dimension significantly longer than the two perpendicular dimensions.

A carrier element adapted to be codelivered with a buttress element may be secured in the bound configuration with one or more binding devices, such as adhesives, tapes, clamps, ties, hooks, clips, sutures, etc. In one or more embodiments, a binding device may hold the carrier element in a bound configuration while, for example, a stapler holding a buttress element having the carrier element attached is passed through an endoscopic passage. After a buttress element is surgically fastened, the binding device or devices may be manipulated (e.g., from the opposite end of the endoscopic passage) to unroll or unbind the carrier element such that the carrier element may be positioned to deliver a prescribed dose of radiation. In one or more embodiments, the binding device is a wire adapted to hold the carrier element in a rolled or bound configuration until it is desired to unroll or unbind the carrier element, after which the wire may be detached from the carrier element and removed (e.g., through an endoscopic passage).

When a carrier element containing radioactive material is unbound, the carrier element may be further secured into place by suturing or otherwise attaching one or more portions of the carrier element to anatomical tissue.

In one or more embodiments, a medical device according to the present disclosure may further include any number of carrier elements having radioactive material attached thereto. For example, a second (or third, fourth, etc.) carrier element having radioactive material attached thereto may be attached to the buttress element. In other words, two or more quantities of radioactive material may be attached to the buttress material via attachment to the buttress material by two or more carrier elements having radioactive material attached thereto. For example, a carrier element having radioactive material (e.g., brachytherapy seeds) may be attached to each of two or more layers of buttress material in a buttress element. In embodiments having two or more carrier elements having radioactive material thereon, each carrier element may be bound by a binding device prior to surgical fastening of the buttress element, wherein each binding device may be manipulated to unbind each carrier element in order to provide a prescribed dose of radiation.

In some embodiments, a medically useful agent may be provided in addition to the buttress elements and radioactive material. The medically useful agent is an agent that has therapeutic, healing, curative, restorative, and/or medicinal properties. For example, the medically useful agent may be provided to, e.g., offset potential radiation-specific injury in tissue proximate the radioactive material, reduce inflammation, increase cell attachment, recruit cells, cause differentiation of the cells to repair the damaged tissue, etc.

Some potentially suitable medically useful agents may include, but are not limited to, e.g., urinary trypsin inhibitor (e.g., UTI, Ulinastatin); 243-aminopropylamino)ethylsulfanyl phosphonic acid (e.g., Amifostine), tissue-inhibitors of metallo-proteinases (e.g., TIMP, TIMP-1, TIMP-3); small molecular inhibitor of transforming growth factor-beta (e.g., SM16); antitransforming growth factor-beta antibody 1D11; recombinant human keratinocyte growth factor (rHuKGF); Interleukin 1-alpha receptor antagonist (IL1RN); Interleukin 6 receptor antagonist (IL6RN); etc.; and combinations thereof. Other examples of some potentially suitable medically useful agents may include, e.g., chemotherapy agents provided as an adjunct treatment to the radiation energy provided by the radioactive material, collagen and insoluble collagen derivatives, hydroxyapatite and soluble solids and/or liquids dissolved therein. Also included are amino acids, peptides, vitamins, co-factors for protein synthesis; hormones; endocrine tissue or tissue fragments; synthesizers; enzymes such as collagenase, peptidases, oxidases; cell scaffolds with parenchymal cells; angiogenic drugs and polymeric carriers containing such drugs; collagen lattices; biocompatible surface active agents, antigenic agents; cytoskeletal agents; cartilage fragments, living cells such as chondrocytes, bone marrow cells, mesenchymal stem cells, natural extracts, tissue transplants, bioadhesives, transforming growth factor (TGF-beta) and associated family proteins (bone morphogenetic protein (BMP), growth and differentiation factors (GDF) etc.), fibroblast growth factor (FGF), insulin-like growth factor (IGF-1) and other growth factors; growth hormones such as somatotropin; bone digesters; antitumor agents; fibronectin; cellular attractants and attachment agents; immuno-suppressants; permeation enhancers; and peptides, such as growth releasing factor, P-15 and the like. Additional medically useful agents that may potentially be used in connection with the buttress elements and radioactive material described herein may be described in US Patent Application Publication No. US 2007/0128243 (Serafica et al.) and/or EP 0957779 B1 (Rayburn et al.).

The medically useful agent may be provided in any suitable form, e.g., as a coating on the staples, buttress material, and/or carrier material. In other embodiments, the medically useful agent may be provided in the form of a solution, gel, etc. that can be provided on the staples, buttress material, and/or carrier material before, during and/or after attachment of the buttress material and/or carrier material. In still other embodiments, the medically useful agent may be incorporated into the staples, buttress material, and/or carrier material (by, e.g., absorption, adsorption, imbibing, etc.).

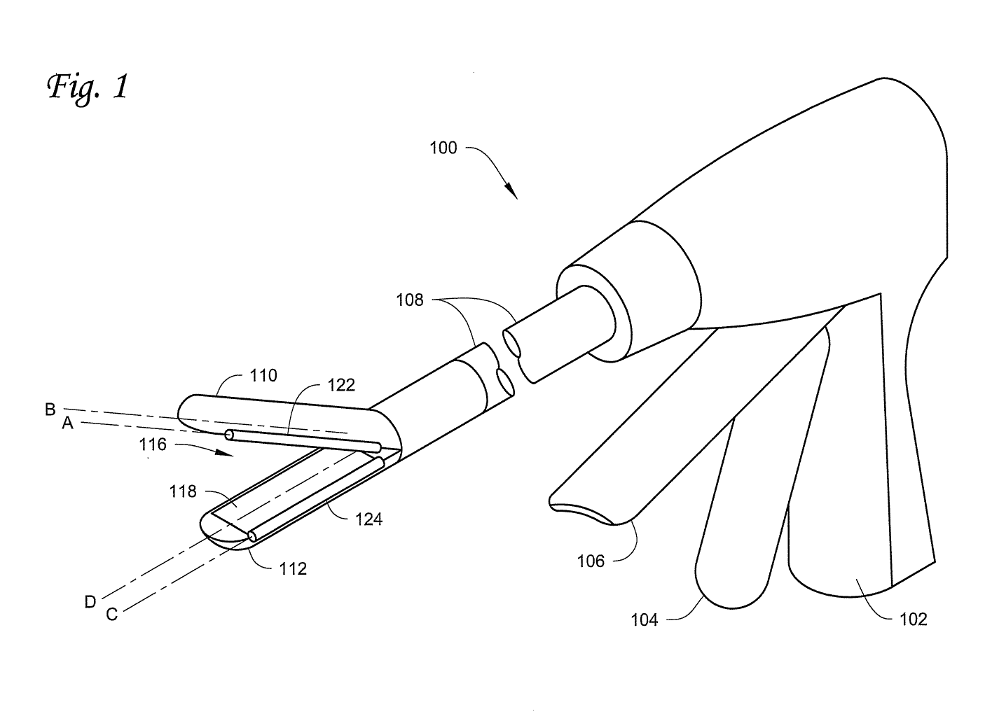

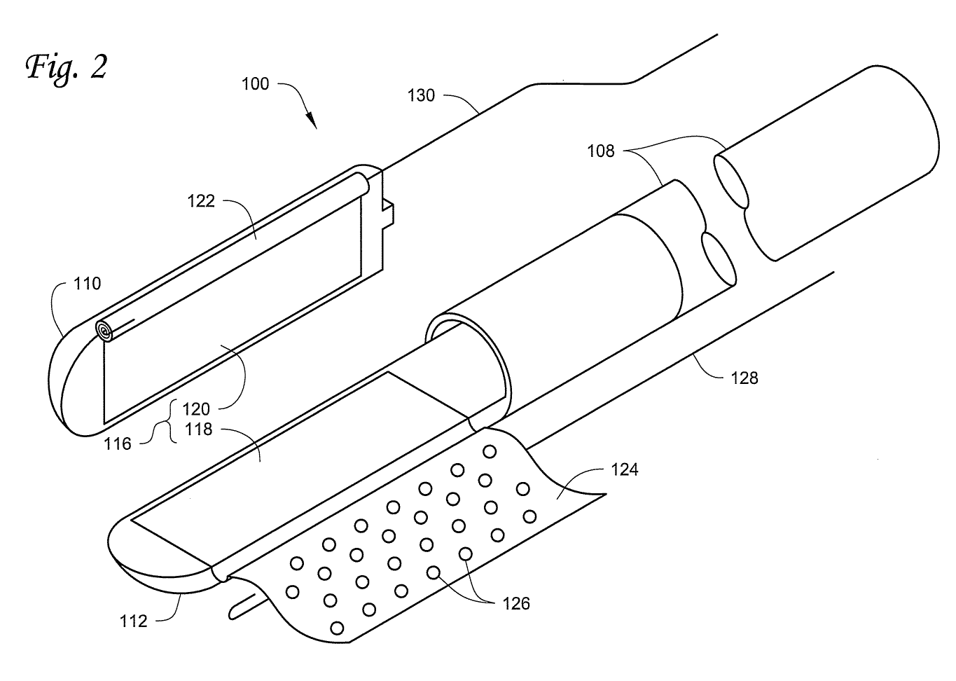

Referring now to FIGS. 1 and 2, one embodiment of a surgical fastening device in the form of a surgical stapler 100 is shown. The surgical stapler in FIG. 1 includes a handle 102, an anvil actuator lever 104, and a staple actuator lever 106. An elongate shaft 108 extends from the surgical stapler to the two stapler clamping elements 110 and 112. In FIG. 1, clamping element 110 can be manipulated to contact clamping element 112 by actuation of the anvil actuator lever 104. Clamping element 112 contains a cartridge of staples (not shown) that may be expelled against clamping element 110 (when clamping element 110 is contacting clamping element 112) by actuation of the staple actuator lever 106.

The surgical stapler 110 includes buttress element 116 having a first layer of buttress material 118 on clamping element 112 and a second layer of buttress material 120 (shown in FIG. 2) on clamping element 110.

FIG. 2 shows an enlarged view of the clamping members 110 and 112. The surgical stapler 100, as depicted in FIG. 2, is displayed in a partially exploded view, with clamping member 110 shown separated from the remainder of the stapler and rotated to expose the buttress material 120 on claiming member 110.

In FIG. 1, a first carrier element 122 is attached to the second layer of buttress material 120 and a second carrier element 124 is attached to the first layer of buttress material 118. Carrier element 122 is shown in a rolled configuration, wherein the roll defines an axis (shown as dashed line A) that is generally aligned with an axis (dashed line B) defined by clamping element 110. Carrier element 124 is also shown in a rolled configuration, wherein the roll defines an axis (dashed line C) generally aligned with an axis (dashed line D) defined by clamping element 112. Each of the carrier elements 122 and 124 include radioactive brachytherapy seeds 126 attached thereto (as depicted in FIG. 2).

The surgical stapler 100 includes a buttress element 116 and two quantities of radioactive brachytherapy seeds (e.g., 126) attached thereto, using bound carrier elements 122 and 124.

When the clamping element 110 is manipulated to contact clamping element 112, the clamping elements contain the buttress element 116 therebetween. In one or more embodiments, anatomical tissue may be placed between the first layer of buttress material 118 and the second layer of buttress material 120, and the clamping element 110 may be manipulated to contact clamping element 112, thereby compressing the anatomical tissue (not shown) and the buttress element 116. The staples may be expelled from the clamping element 112 through the anatomical tissue and the buttress element 116 to surgically fasten the anatomical tissue located between clamping elements 110 and 112.

During deployment, the carrier elements 122 and 124 may be unrolled, taking a substantially flat, sheet-like configuration. In FIG. 2, carrier element 122 is in a rolled configuration and carrier element 124 is in an unrolled, substantially flat, sheet-like configuration. The brachytherapy seeds 126 are depicted as arranged on carrier element 124 in a repeating pattern.

Carrier element 122 is shown in FIG. 2 in a rolled configuration, held in such a configuration by a first binding device 130. The first binding device 130 is shown in FIG. 2 as passing through the rolled carrier element 122 and binding the carrier element with a hook on the end of the first binding device 130. FIG. 2 also shows a second binding device 128, which has been manipulated to unroll carrier element 124. After a carrier element (122 and 124) is unrolled or unbound, the binding device may be removed from the resection site.

After the buttress materials 118 and 120 and attached carrier elements 122 and 124 have been deployed, it may be beneficial to further secure the carrier elements 122 and 124 such that they lie against the buttress material 118 and 120 by, e.g., adhesives, tapes, clamps, ties, hooks, clips, sutures, etc. Appropriately securing the carrier elements 122 and 124 may be beneficial in providing a prescribed dose of radiation.

The buttress materials and carrier materials may be provided in any form that is suitable for delivery using a stapler as described herein. The buttress material and/or carrier elements may be attached to the delivery device (e.g., a stapler, etc.) by any suitable technique, e.g., clamps, ties, hooks, clips (e.g., Weck clips, etc.), sutures, adhesives, tapes, etc. Examples of some potentially suitable stapler-based delivery systems for buttress material may be described in one or more of the following documents: U.S. Pat. No. 5,752,965 (Francis et al.); U.S. Pat. No. 5,810,855 (Rayburn et al.); U.S. Pat. No. 6,939,358 (Palacios et al.); US Patent Application Publication US 2004//0093029 (Zubiket al.); etc.

Although one illustrative embodiment of a stapler 100 is depicted in connection with FIG. 1, it should be understood that the buttress material (and carrier material, if used) can be delivered by many other potential devices, some of which may be described in the patent documents cited herein. Furthermore, the staplers and any buttress material may be delivered to the tissue to be buttressed by any suitable technique, e.g., open surgery, laparoscopic surgery, endoscopic surgery (e.g., Natural Orifice Translumenal Endoscopic Surgery (NOTES)), remote surgery (using, e.g., robotic systems); etc.

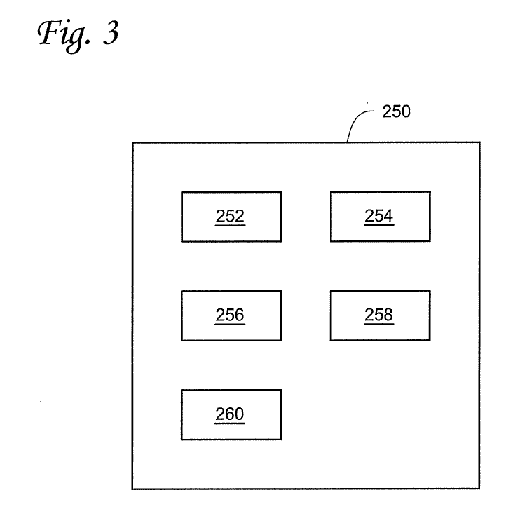

The staples, buttress material, radioactive material, carrier material, and/or one or more medically useful agents as described herein may, in some embodiments, be provided in the form of a kit configured for use with one or more staplers. FIG. 3 is a schematic representation of one such kit which includes components that may be provided in a sealed package 250. Among the components that may be included in the kit are staples 252, buttress material 254, radioactive material 256, carrier material 258, and medically useful agent 260. Some of these components may optionally be included in the kit. For example, some kits may be limited to buttress material 254 and radioactive material 256, while other kits may include only radioactive material 256 and carrier material 258 (with, e.g., the carrier material 258 being configured for use with separately-supplied buttress material). As discussed herein, two or more of these components may be provided integrally with each other although they are depicted as separate components within the kit. For example, the radioactive material 256 may be incorporated into the staples 252, the buttress material 254, and/or the carrier material 258. In other examples, the medically useful agent 260 may be provided integrally with the staples 252, buttress material 254, and/or carrier material 258.

As used herein and in the appended claims, the singular forms “a,” “and,” and “the” include plural references unless explicitly limited to the singular form or the context clearly dictates otherwise.

All references and publications cited herein are expressly incorporated herein by reference in their entirety into this disclosure. Illustrative embodiments of this invention are discussed and reference has been made to possible variations within the scope of this invention. These and other variations and modifications in the invention will be apparent to those skilled in the art without departing from the scope of the invention, and it should be understood that this invention is not limited to the illustrative embodiments set forth herein. Accordingly, the invention is to be limited only by the claims provided below and equivalents thereof.

1. A kit for buttressing tissue at an internal body location, the kit comprising:
one or more sheets of buttress material in a package, wherein the buttress material is configured for delivery and attachment to tissue using a stapler;
radioactive material in the package, wherein the radioactive material is configured for attachment to the one or more sheets of buttress material.

2. A kit according to claim 1, wherein the radioactive material is provided integrally with the buttress material.

3. A kit according to claim 1, wherein the radioactive material is incorporated into one or more fibers in the buttress material.

4. A kit according to claim 1, wherein the radioactive material is separate from the buttress material.

5. A kit according to claim 1, wherein the kit comprises carrier material, and wherein the radioactive material is provided integrally with the carrier material.

6. A kit according to claim 5, wherein the radioactive material is incorporated into one or more fibers in the carrier material.

7. A kit according to claim 1, wherein the kit further comprises staples.

8. A kit according to claim 7, wherein the radioactive material is provided integrally with the staples.

9. A kit according to claim 1, wherein the kit further comprises a medically useful agent.

10. A kit according to claim 9, wherein the medically useful agent is provided integrally with the buttress material.

11. A kit according to claim 1, wherein the kit further comprises staples and carrier material, and wherein the radioactive material is provided integrally with the carrier material.

12. A kit according to claim 11, wherein the kit further comprises a medically useful agent.

13. A kit according to claim 12, wherein the medically useful agent is provided integrally with the buttress material.

14. A kit according to claim 12, wherein the medically useful agent is provided integrally with the staples.

15. A kit according to claim 12, wherein the medically useful agent is provided integrally with the carrier material.

16. A surgical fastening device comprising:
a buttress element; and
radioactive material attached to the buttress element.

17. A device according to claim 16, wherein the buttress element is attached to a surgical fastening instrument.

18. A device according to claim 17, wherein the surgical fastening device comprises a surgical stapler and wherein the buttress element is adapted for delivery to a staple line formed by the stapler.

19. A device according to claim 16, wherein the buttress element comprises two or more layers of buttress material.

20. A device according to claim 16, wherein the radioactive material comprises a radioactive isotope.

21. A device according to claim 16, wherein the radioactive material is a radioactive isotope of an element selected from the group consisting of iodine, palladium, or a combination thereof.

22. A device according to claim 16, wherein the radioactive material is a radioactive isotope selected from the group consisting of iodine-125, palladium-103, and combinations thereof.

23. A device according to claim 16, wherein the radioactive material comprises a seed or a radioactive fiber.

24. A device according to claim 16, wherein the radioactive material is attached to a carrier element.

25. A device according to claim 25, wherein the carrier element is selected from the group consisting of vicryl mesh, polygluconate, bovine pericardium, small intestinal submucousa, and combinations thereof.

26. A device according to claim 24, wherein the carrier element is attached to the buttress element.

27. A device according to claim 24, wherein the radioactive material comprises seeds attached to the carrier element.

28. A device according to claim 24, wherein the radioactive material is distributed on the carrier element to deliver a prescribed radiation dose.

29. A device according to claim 16, wherein the radioactive material is attached to the buttress element in a longitudinal configuration having a first axis having a dimension significantly longer than the dimensions in axes transverse to the first axis.

30. A device according to claim 24, wherein the carrier element is bound, and wherein the bound carrier element is rolled or folded.

31. A device according to claim 30, wherein the bound carrier element is mechanically bound with a binding device, wherein the binding device is optionally selected from the group consisting of a clamp, tie, hook, clip, or a combination thereof; and wherein the binding device is optionally removable from the carrier element; and further wherein the binding device may be manipulated to release the bound carrier element such that the carrier element may comprise a flat, sheet-like configuration.

32. A device according to claim 16, wherein the radioactive material is provided in two separate and discrete quantities.

33. A method buttressing a staple line and providing radiation therapy, the method comprising:
attaching buttress material to selected tissue using staples;
attaching radioactive material to the buttress material, wherein radiation therapy is provided to tissue proximate the buttress material.

34. A method according to claim 33, further comprising providing a medically useful agent proximate the buttress material.
